(19) United States
(12) Patent Application Publication
(10) Pub. No.: US 2024/0398698 A1
(43) Pub. Date: Dec. 5, 2024

(54) CONAZOLANTIFUNGAL KETOE IN SOLUTION OR SUSPENSION FOR USE IN THE HUMAN EAR

(71) Applicant: David Alessi, Beverly Hills, CA (US)

(72) Inventor: David Alessi, Beverly Hills, CA (US)

(21) Appl. No.: 18/204,812

(22) Filed: Jun. 1, 2023

(51) Int. Cl. A61K 9/00; A61K 31/496; A61K 45/06; A61K 47/10; A61P 31/10

(52) U.S. Cl. CPC A61K 9/0046; A61K 31/496; A61K 45/06; A61K 47/10; A61P 31/10

(57) ABSTRACT

A pharmaceutical solution or suspension of 0.01% to 50% ketoconazole is formulated as an ear drop for use in the human ear for the treatment of otomycosis. The ear drop will be formulated with and without anti-bacterial agents including neomycin, polymyxin, ofloxicin, ciprofloxin, tobramycin, moxafloxin, acetic acid, isopropyl alcohol, and gentamycin all in varying concentrations admixed with a solvent (mainly water) for the treatment of infectious otitis externa where the pathogen isn't clear. It will also be formulated with and without corticosteroids for instances where there is significant ear canal edema.

### FIELD OF INVENTION

The present invention relates to a composition (ear drop) for the topical treatment of fungal ear infections (known as otomycosis) delivered through the ear canal in humans, and more particularly, to an improved method of treating otomycosis than is currently available.

### BACKGROUND OF THE INVENTION

Ear infections are common in humans and include bacterial, fungal, and yeast infections and can involve the ear canal (otitis externa) and middle ear (otitis media) and sometimes both if the eardrum is not intact. There are very few compounds available to topically treat otomycosis. Acetic acid (Burow's solution) is commonly used to slow the growth of the fungus but is not a targeted anti-fungal. Clotrimazole drops have been reported but are not available in the U.S. In the office, gentian violet is used topically. Most often, oral agents are used because of the lack of satisfactory ototopicals (ear drops). The use of oral agents has a higher risk of complications such as toxicity to the liver and kidneys, interaction with other medications (antibiotics and cholesterol lowering medications are common), and more severe systemic allergic reactions. Additionally, anti-fungal oral medications often cannot penetrate the infected tissues because of the unique properties of fungal infections.

U.S. Pat. No. 7,732,450, issued to Whitefield et al., discloses a pharmaceutical composition suitable for topical application comprising ketoconazole in a solution which comprises: (i) 13 to 50 wt % water; (ii) 45 to 85 wt % of an alcohol having a boiling point of less than 100° C. ; and (iii) 2 to 30 wt % of a non-volatile, water miscible, non-ionic surface active agent; the ketoconazole being present in an amount of 0.5 to 3 wt % relative to components (i), (ii) and (iii).

U.S. Pat. No. 9,839,608, issued to Albert et el., discloses a method for treating ear infections in animals includes forming a liquid composition having an antifungal agent, an antibiotic agent, a steroidal anti-inflammatory agent, an anhydrous organic acid, liquid dehydrated alcohol, and propylene glycol. The treatment method includes applying several drops of such composition inside the ear of the animal at least twice per day to the affected area of the animal's ear until the ear infection has been cured. The antifungal agent may include ketoconazole. The antibiotic agent may include ofloxacin. The steroidal anti-inflammatory agent may include triamcinolone diacetate. The anhydrous organic acid may include citric acid.

U.S. Pat. No. 7,374,772, issued to Bommarito. discloses a topical skin preparation for treatment of fungal infections of the skin and nails. The preparation comprises triacetin in combination with an antifungal agent. In a preferred form, the preparation further comprises, a fatty acid source such as a fish oil. In a preferred embodiment, cod liver oil and tolnaftate are used in combination with triacetin. The concentrations of these constituents are 96.0-99.0% concentration triacetin, 0.5-3.0% concentration tolnaftate and 0.5-1.0% concentration cod liver oil, in one preferred embodiment. Other compounds, such as ethyl alcohol, amino acids such as n-acetylcysteine, and herbal additives may also be added to the preparation. Further, other antifungal agents such as nystatin, clortimazole, econazole, ketoconazole, miconazole, solconazole, oxiconizole, naftifine, terbinafine, and butenafine, for example, may be substituted for the antifungal agent tolnaftate. The preparation is effective in treating immune compromised patients and those with diabetes, as well as relatively healthy persons.

U.S. Pat. No. 6,130,254, issued to Fisher et at., discloses methods for ameliorating various effects of UVA and UVB radiation from the sun, comprising administering compositions including an ingredient that prevents photoaging from MED and subMED radiation, such as a retinoid, certain other compounds (such as N-acetylcysteine, 2-furildioxime, and vitamin C) and optionally other MMP inhibitors such as tetracyclines and/or compounds that inhibit the P-450-mediated metabolism of retinoids such as ketoconazole and other azole compounds. In the method, the composition is applied prior to exposure to the sun; depending upon the ingredients used in the composition, application should be from 7 to 48 hours prior to exposure. Compounds that prevent erythema (skin reddening, sunburn) do not necessarily protect against UV-mediated elevation of MMP levels and activity, and similarly compounds that prevent UV-mediated elevation of MMP levels and activity are not necessarily effective against UV-induced erythema.

U.S. Pat. No. 6,080,744, issued to Ayon-Covarrubias discloses a topical mycological treatment composition for medical, veterinarian, or dental use contains as active ingredients clotrimazole, ketoconazole, micanazole, nystatin, tolnaftate, propionic acid, sodium propionate, undecelynic acid, and zinc undecelynate. These ingredients are contained in a natural cream base, and the base may also contain an anti-inflammatory agent and an antibacterial agent.

It is the objective of the instant invention is to present a known anti-fungal agent (ketoconazole) directly to the infection. Ketoconazole is known to be effective in fungal infections when taken orally. Also, it has been used successfully as a topical agent for skin fungal infections. Although not intuitive, the use of ketoconazole for ear canal infections in humans as an ear drop is expected to be efficacious. It is a further objective to provide the formulation in both a suspension and in solution.

Additionally, frequently it is not obvious if an ear canal infection is fungal, yeast, bacterial, or mixed. Use of an anti-bacterial agent will make fungal infections worse. Conversely, use of an anti-fungal agent can make a bacterial infection worse. Although not previously described for humans, it is a still further objective to provide for the combined use of ketoconazole and an anti-bacterial agent to address the dilemma of the type of ear infection.

As the ear canal can become swollen from ear infections, and as topical steroids are well-known to reduce swelling in the ear canal, it is a final objective of the present invention to provide for a combination of the anti-fungal agent with topical steroids to allow the anti-infectious agent in the drops to be effective in mixed infection cases.

While some of the objectives of the present invention are disclosed in the prior art, none of the inventions found include all of the requirements identified.

### SUMMARY OF THE INVENTION

The present invention addresses all of the deficiencies of prior art anti-fungal treatments and satisfies all of the objectives described above.

Briefly described, and accordance with the preferred embodiments thereof, the present invention relates to a method using a liquid composition (ear drop) which is effective in the topical treatment of human external canal or middle ear infections (otomycosis), and its manner of use to treat ear fungal/yeast infections. The method uses a composition that includes an antifungal agent, ketoconazole. Alternative embodiments include the simultaneous use of an anti-bacterial agent or anti-inflammatory steroid. The active agent will be in a liquid form so that it may be used as an ear drop that can be introduced into the ear canal. The formation of the liquid may include water, isopropyl alcohol, dehydrated alcohol, and propylene glycol.

The antifungal agent of the composition is a known antifungal imidazole called ketoconazole. Ketoconazole is used because of its efficacy as an oral systemic agent, strong safety profile and use as a dermatologic compound. The concentration of ketoconazole in the composition will be in a range of 0.01% to 50%

The antibacterial agent may include an antibiotic, or a compound shown to inhibit bacterial growth such as acetic acid. The antibiotics include agents that have already been used in the treatment of ear infections topically such as ofloxacin, ciprofloxacin, moxifloxacin, sulfa agents, tobramycin, and gentamycin. The concentration of the antibiotic will be 0.001% to 50%. Acetic acid will be used in a concentration of 0.01% to 10%.

The preferred embodiment will be an ear drop with ketoconazole as the antifungal agent that adequately treats otomycosis without significant residue left over time and with fairly constant concentration in all areas of the application site. It is anticipated that a solution will be the preferred vehicle.

In the preferred embodiment, formulation methods are practiced to ensure that the resulting composition is homogeneous. In this regard, the preferred formulation dissolves the antifungal agent with or without the additional steroid agent in liquid dehydrated alcohol to form a first solution. The antibiotic agent is separately dissolved in liquid propylene glycol to form a second solutions. The first and second solutions are combined to form a third solution and this third solution is then heated. The container used to heat the third solution is covered, as by an impervious plastic film, and the third solution is preferably stirred during heating. The step of covering the container during prevents the dehydrated alcohol from evaporating and helps keep the steroidal anti-inflammatory agent in solution for a more homogeneous solution. The solution is cooled back to room temperature preferably with stirring during the cooling phase. The solution will then be placed in a bottle to provide for dispensing uniform size ear drops.

The present invention also relates to the method of treating fungal, yeast or mixed ear infections in humans by formulating a composition of the type described above, and applying the composition inside the ear canal of the patient at least twice per day until the infection has been cleared.

### DETAILED DESCRIPTION OF THE PREFERRED EMBODIMENT

A composition for the topical treatment of otomycosis in humans, formulated in accordance with the preferred embodiment of the present invention, includes ketoconazole and may include the following additional active ingredients: an anti-infective, an antibiotic, and a steroidal anti-inflammatory agent. The preferred embodiment will also include solvents such as dehydrated alcohol, water, propylene glycol, dimethyl sulfoxide or other solvents for maintaining the forgoing active ingredients in solution.

An antifungal agent is generally defined as a drug that selectively eliminates fungal and usually yeast pathogens from a host human with minimal toxicity to the host. Some antifungal agents are selective in the types of fungal infections which they attack, while other antifungal agents are effective to treat a broad spectrum of fungal infections. The composition of the present invention preferably utilizes a topical form of ketoconazole which has the ability to treat fungal and yeast infections caused by organisms such as Candida Albicans, Aspergillus niger, Aspergillus species, Aspergillus fumigatus, and Candida parapsilosis in the ear canal and middle ear. Among the major groups of antifungal agents having applicability to the present investigation are the polyenes, the azoles, the allylamines, the morpholines, and antimetabolites, as well as a combination of one or more of such antifungal agents. Ketoconazole, an imidazole, is a broad spectrum antifungal agent that can treat the most common fungal or yeast pathogens found in human otomycosis. It has an excellent safety profile especially as a dermatologic topical agent. When ingested, there are risks with hepatotoxicity, androgen suppression and anaphylactic reactions. These risks are rare but nonetheless minimized with a topical ear drop formulation.

When preparing the composition, the antifungal agent is a solid at room temperature and will be provided in powder form and dissolved in dehydrated alcohol. Ketoconazole is poorly soluble in water but is soluble in alcohol. The preferred initial concentration will be 5% of the ketoconazole. Additionally, a suspension of ketoconazole at 5% may be needed with water as the liquid vehicle. The latter may be required since alcohol is ototoxic in the inner ear and painful in the middle ear.

An antibiotic or antibacterial agent is generally defined as a drug that selectively eliminates bacteria from a host with minimal toxicity to the host. Although the invention is meant as a stand-alone product (ie ketoconazole alone), there are occasionally mixed infections or unclear infections where the practitioner is not certain if it is a bacterial or fungal infection. In these cases, ketoconazole combined with an anti-infective agent or an antibiotic would be beneficial. As with the antifungal agent, the antibiotic agent is preferably a broad-spectrum type antibiotic effective in killing a wide range of bacteria. Acetic acid is an antibacterial agent and has antifungal properties. With the formulation of ketoconazole, an antibacterial preparation with acetic acid will be made since the antifungal properties of acetic acid may be complimentary with that of ketaconazole. However, it is anticipated that a formulation with an agent safe for the middle ear will be made since acetic acid cannot be placed in the middle ear. An antibiotic with an excellent safety record as a middle ear topical agent is ofloxacin. Other agents that may be used are ciprofloxacin, moxifloxacin, tobramycin, gentamycin, neomycin, polymyxin B, and sulfa agents. Other suitable antibiotic agents which may be used in practicing the present invention may be found in “Drug Facts and Comparisons, 2002,” published by Facts and Comparisons.

The antibiotic agent ofloxacin is a solid and will be used in powder form. Although soluble in water if prepared properly, propylene glycol or dimethyl sulfoxide will be used as a solvent in that it can be combined with the ketoconazole and alcohol compound. Alternatively, if studies show that ketoconazole in suspension is as effective as the solution form, then a purely aqueous form will be used. Since ketoconazole is not soluble in acetic acid, the suspension studies with be especially applicable for the formulation with acetic acid. The concentration of ofloxacin to be used is 0.3%.

Anti-inflammatory steroids have been used as topical agents in a whole host of ear infections previously. Although the invention will be a stand-alone agent using ketaconazole, a variant of the invention is a combination of this agent with a steroid. Steroids are useful in fungal infections to reduce the swelling in the ear canal that can occur with ear infections. Suitable steroid anti-inflammatory agents, also known as adrenocortical steroids, for use with the composition of the present invention include triamcinolone, betamethasone, hydrocortisone, dexamethasone, and combinations thereof. Other suitable steroidal anti-inflammatory agents which may be used in practicing the present invention may be found in “Drug Facts and Comparisons, 2002,” published by Facts and Comparisons. It is anticipated that several agents will be tested in the presence of an acute infection to assess clinical efficacy.

The steroid agent will be used in powder form when the composition is prepared using a steroid. The preferred compound will used dexamethasone. The steroidal agent will be dissolved in dehydrated alcohol since it is practically insoluble in water. Alternatively, a suspension will be prepared. Dexamethasone is currently used in ear drops in a suspension form and is efficacious. The expected concentration of dexamethasone will be 0.1%.

Dehydrated alcohol will be the solvent for the solution form of the invention with or without the additions of antibiotics or steroids. Dehydrated alcohol is a nearly pure form of ethanol with minimal amounts of residual water. The pH is typically lower than 7.0 but may need to be lower depending on the additives used. If the alcohol needs to be made more acidic, hydrochloric acid or various organic acids such as the anhydrous forms of citric acid, glycolic acid, and tartaric acid may be used. Most acids are relatively stable in room temperature ethanol.

The presence of the organic anhydrous acid in the composition of the present invention is believed to provide a beneficial, unexpected effect of increasing the antibacterial effectiveness of the compound. Additionally, it has been found that the inclusion of the anhydrous acid also helps to keep the antifungal and steroidal anti-inflammatory agent fully dissolved in solution. Hydrous acids, such as acetic acid, tend to exhibit the opposite effect. Accordingly, the composition of the present invention need not be agitated or shaken to a significant extent prior to use and the dispensed dosages tend to be more uniform.

If the suspension if used, there will be settling of the agent(s). Although a nuisance, this is typically not an issue. There are several current topicals that required shaking prior to use. It is expected that the suspension will be the most applicable formulation of the invention for middle ear use.

In preparing the solution formulation, propylene glycol (or dimethyl sulfoxide) will be used to dissolve the formulation with an antibiotic. After combining the dehydrated alcohol solution (containing the dissolved antifungal agent, possibly the anti-inflammatory steroid and possibly the anhydrous acid) and the propylene glycol solution (containing the dissolved antibiotic agent), the composition is continuously stirred and heated, generally at least until the solution becomes clear and homogeneous. The solution is the cooled to room temperature while continuously stirring. An impervious plastic film or other seal is used to cover the open end of the container used to hold the solution during the heating and cooling processes. The solution will then be placed in 10 ml ear drop vials for use by patients with the appropriate prescription from the practitioner and, of course, after FDA approval.

### Examples of Clinical Use

Example 1. A patient presented with an acute fungal ear infection. The ear canal is debrided and the ear drum is intact. The patient is given a prescription for ketoconazole ear drops 5% in solution to use 3-5 drops in the affected ear twice a day. The patient returns one week later and the infection is completely cleared. The ketoconazole ear drops were prepared by dissolving 5.0 grams of ketoconazole (medical grade purchased from Janssen Pharmaceutica Products in New Jersey) in 100 cc of anhydrous alcohol. A 10 cc bottle was dispensed to the patient.

Example 2. A patient presented with otomycosis. The ear canal is debrided and the canal is not swollen. The doctor is unclear if the ear drum is intact. The patient is given a prescription for ketoconazole ear drops 5% in suspension to use 3-5 drops in the affected ear twice a day. The patient returns one week later and the infection is completely cleared. The ketoconazole ear drops were prepared by mixing 5.0 grams of ketoconazole in 100 cc sterile 0.9% saline with benzalkonium chloride (0.0025%). Hydrochloric acid and sodium hydroxide are added to adjust the pH to 6.5+/−0.5. A 10 cc bottle was dispensed to the patient.

Example 3. A patient presented with otomycosis. The ear canal is debrided and the canal is very swollen. The patient is given a prescription for ketoconazole 5%/dexamethasone 0.1% in solution ear drops to use 3-5 drops in the affected ear twice a day. The patient returns one week later and the infection is completely cleared. The ketoconazole ear drops were prepared by dissolving 5.0 grams of ketoconazole and 0.1 gram of dexamethasone were dissolved in 100 cc of anhydrous alcohol. A 10 cc bottle was dispensed to the patient.

Example 4. A patient presented with otomycosis. The ear canal is debrided, the canal is swollen, and the doctor is unclear if it there is a bacterial component. The patient is given a prescription for ketoconazole 5%/dexamethasone 0.1%/ofloxacin 0.3% in solution ear drops to use 3-5 drops in the affected ear twice a day. The patient returns on week later and the infection is completely cleared. The ear drop mixture was prepared by dissolving 5.0 grams of ketoconazole and 0.1 grams of dexamethasone in 50 cc of anhydrous alcohol. 30 milligrams of ofloxacin powder was dissolved in 50 cc of propylene glycol. The two mixtures were combined and a 10 cc bottle was given to the patient.

Example 5. A patient presented with otomycosis. The ear canal is debrided and the canal is swollen. The doctor is unclear if the ear drum is intact. The patient is given a prescription for ketoconazole 5%/dexamethasone 0.1% ear drops in suspension to use 3-5 drops in the affected ear twice a day. The patient returns one week later and the infection is completely cleared. The ketoconazole ear drops were prepared by mixing 5.0 grams of ketoconazole and 0.1 grams of dexamethasone in 100 cc sterile 0.9% saline with benzalkonium chloride (0.0025%). Hydrochloric acid and sodium hydroxide are added to adjust the pH to 6.5+/−0.5. A 10 cc bottle was dispensed to the patient.

Example 6. A patient presented with otomycosis. The ear canal is debrided and the canal is not swollen. The doctor is unclear if the ear drum is intact. Furthermore, it is unclear if there is a bacterial component. The patient is given a prescription for ketoconazole 5%/dexamethasone 0.1%/ofloxacin 0.3% ear drops in suspension to use 3-5 drops in the affected ear twice a day. The patient returns one week later and the infection is completely cleared. The ketoconazole ear drops were prepared by mixing 5.0 grams of ketoconazole in 100 cc sterile 0.9% saline with benzalkonium chloride (0.0025%). Hydrochloric acid and sodium hydroxide are added to adjust the pH to 6.5+/−0.5. A 10 cc bottle was dispensed to the patient.

The Antifungal Ketoconazole in Solution or Suspension For Use In The Human Ear has been described with reference to particular embodiments. Other modifications and enhancements can be made without departing from the spirit and scope of the claims that follow.

1. A method of topically treating fungal ear infection in humans (otherwise known as otomycosis) with ear drops, with said method comprising the steps of:
forming a liquid composition herein called “ear drops” (either a solution or suspension) containing ketoconazole; and
applying the composition inside the ear canal of a human patient with otomycosis until cured.

2. The method of claim 1, further comprising the step of adding a component to the composition selected from the group composed of:
water, isopropyl alcohol, dehydrated alcohol, propylene glycol or other liquid vehicle.

3. The methods of claim 1 comprising thew further step of adding an antibiotic agent to the composition selected from the group composed of:
ofloxacin, ciprofloxacin, tobramycin, gentamycin, moxifloxacin. sulfa agents, acetic acid and other antibiotic agents.

4. The methods of claim 2 comprising thew further step of adding an antibiotic agent to the composition selected from the group composed of:
ofloxacin, ciprofloxacin, tobramycin, gentamycin, moxifloxacin. sulfa agents, acetic acid and other antibiotic agents.

5. The method of claim 1 comprising the further step of adding an anti-inflammatory steroid or cortisone agent to the composition.

6. The method of claim 2 comprising the further step of adding an anti-inflammatory steroid or cortisone agent to the composition.
